(19) United States
(12) Patent Application Publication
(10) Pub. No.: US 2009/0317404 A1
(43) Pub. Date: Dec. 24, 2009

(54) REDUCTION AND PREVENTION OF CELL-ASSOCIATED HIV TRANSEPITHELIAL MIGRATION, MICROBIDES AND OTHER FORMULATIONS AND METHODS

(75) Inventor: Richard Markham, Columbia, MD (US)

Correspondence Address: EDWARDS ANGELL PALMER & DODGE LLP, P.O. BOX 55874, BOSTON, MA, 02205, US

(73) Assignee: Johns Hopkins University, Baltimore, MD (US)

(21) Appl. No.: 12/083,582

(22) PCT Filed: Oct. 13, 2006
(86) PCT No.: PCT/US2006/040360

§ 371 Date: Mar. 3, 2009

Related U.S. Application Data
(60) Provisional application No. 60/726,738, filed on Oct. 14, 2005.

(51) Int. Cl. A61K 39/42; C07K 16/00; C12N 1/21

(52) U.S. Cl. 424/159.1; 530/387.1; 435/252.3

(57) ABSTRACT

A vaginal microbicide including scFv-type antibodies reduces or prevents transepithelial HIV transmission. As the antibodies, anti-CD 18 and/or anti-CD 11 antibodies (of the scFv type) are used. Preferably, anti-ICAM antibody also is used. The antibodies may be delivered to the to-be-protected epithelium using a bacterial delivery system such as a lactobacillus bacterial vehicle. HIV transepithelial transmission can thereby be reduced or prevented, including prevention of initial infection.

### BACKGROUND OF THE INVENTION

1. Field of the Invention

The invention generally relates to methods of combating the sexual transmission of HIV infection. In particular, the invention provides methods for using antibodies to CD18 to inhibit human immunodeficiency virus (HIV) from crossing the cervical, vaginal, gastrointestinal, rectal, colonic and oral epithelium.

2. Background of the Invention

HIV-1 infections are acquired most often through sexual contact, with the majority of sexual transmission of HIV-1 worldwide occurring as a result of heterosexual contact (Louria, D. B., J. H. Skurnick, P. Palumbo, J. D. Bogden, C. Rohowsky-Kochan, T. N. Denny, and C. A. Kennedy, 2000, HIV heterosexual transmission: a hypothesis about an additional potential determinant, Int J Infect Dis 4:110-6; Skurnick, J. H., C. A. Kennedy, G. Perez, J. Abrams, S. H. Vermund, T. Denny, T. Wright, M. A. Quinones, and D. B. Louria, 1998, Behavioral and demographic risk factors for transmission of human immunodeficiency virus type 1 in heterosexual couples: report from the Heterosexual HIV Transmission Study, Clin Infect Dis 26:855-64.) Women of childbearing age are at the greatest risk for HIV-1 infection (Davis, S. F., D. H. Rosen, S. Steinberg, P. M. Wortley, J. M. Karon, and M. Gwinn, 1998, Trends in HIV prevalence among childbearing women in the United States, 1989-1994, J Acquir Immune Defic Syndr Hum Retrovirol 19:158-64; Wortley, P. M., and P. L. Fleming, 1997, AIDS in women in the United States, Recent trends, Jama 278:911-6), which has resulted in a corresponding increase in HIV-1 infection of women, newborns and infants worldwide. For example, mechanisms of sexual transmission of HIV-1 to women are being studied for potential targets for HIV-1 prevention in women. Also, potential approaches for rectal uses in men and women are considered to prevent transmission via that route.

Microbicides are a potentially woman-controlled preventive intervention that may be used to reduce the incidence of new HIV-1 infections. Even a partially effective microbicide may have a significant impact on the HIV epidemic; it has been estimated that if only 20 percent of women in 73 low-income countries used a 60-percent efficacious microbicide for half of all otherwise unprotected sex acts, 2.5 million HIV infections would be averted over three years in women, men, and infants (Watts, C., W. Thompson, and L. Heise, 1998, Presented at the International Conference on AIDS, Geneva.)

A variety of potential anti-HIV-1 microbicides are currently in advanced stages of development and clinical testing. However, these microbicides entail costs of production that would make them impractical for use in the most at risk populations. Also, the microbicides currently being tested present several drawbacks including reduced effectiveness against some CCR5-utilizing isolates, disruption of the normal flora of the genitourinary tract, toxicity for the genital epithelium, carcinogenic potential, and undemonstrated efficacy against cell-associated virus, reviewed in D'Cruz, O. J., and F. M. Uckun, 2004, Clinical development of microbicides for the prevention of HIV infection, Curr Pharm Des 10:315-36. Use of antibodies as microbicides, such as the vaginally applied anti-gp120 antibodies used to protect against transmission of SHIV in macaques, may avoid the toxicity problems associated with many chemical compounds (Veazey, R. S., R. J. Shattock, M. Pope, J. C. Kirijan, J. Jones, Q. Hu, T. Ketas, P. A. Marx, P. J. Klasse, D. R. Burton, and J. P. Moore, 2003, Prevention of virus transmission to macaque monkeys by a vaginally applied monoclonal antibody to HIV-1 gp120, Nat Med 9:343-6)

One difficulty in designing chemically- or antibody-based microbicides targeting cell-free transmission of HIV-1 is the high degree of variability of viral surface epitopes. This difficulty can be avoided by targeting epitopes of the host cell proteins involved in cell-associated and/or cell-free virus transmission. Previously, host ICAM-1 has been identified as a potential target for antibody-based microbicide development. Anti-ICAM-1 has been shown to disrupt transmission of cell-associated HIV-1 both in vitro across a model cervical epithelial cell monolayer, and in vivo in a HuPBL-SCID mouse model of HIV-1 transmission. Notably, results in the mouse model demonstrated that engagement of ICAM-1 on the murine epithelium was necessary for blocking transmission, and that blocking only ICAM-1 on the infected human PBMC inoculum was insufficient for blocking transmission (Chancey, C. J., K. V. Khanna, J. F. Seegers, G. W. Zhang, J. Hildreth, A. Langan, and R. B. Markham, 2006, Lactobacilli-expressed single-chain variable fragment (scFv) specific for intercellular adhesion molecule 1 (ICAM-1) blocks cell-associated HIV-1 transmission across a cervical epithelial monolayer, J Immunol 176:5627-36).

However, despite newer methods of antibody production, the cost for such a microbicide necessarily involving passive administration of antibody is likely to be prohibitive.

As background, there is mentioned:

U.S. Pat. No. 6,566,095 (May 20, 2003) to Markham et al. (Johns Hopkins University), for “Compositions and methods for preventing transepithelial transmission of HIV.”

Although it would be advantageous to provide antibodies capable of blocking sexual transmission of a broad array of HIV-1 variants in an economically and clinically feasible manner, such antibodies have not yet been provided before this invention. A major problem with any approach that targets viral antigens has been that in the case of HIV, these are highly variable and mutate frequently.

### SUMMARY OF THE INVENTION

The present invention exploits the discovery by the inventors that anti-CD18 antibodies disrupt HIV-1 transmission across the epithelium. Anti-CD18 antibodies are thus useable in a microbicide to prevent or attenuate HIV-1 transmission across the cervical epithelium, vaginal epithelium, gastrointestinal epithelium, rectal epithelium, colonic epithelium and oral epithelium; a method against HIV-1 infection may thereby be provided.

In addition, the inventors have discovered that anti-CD18 antibodies may be used in combination with antibodies to ICAM-1. Further, the concentration of antibodies with which this effect is produced is sufficiently low to be obtainable in a clinical setting.

In addition, the invention also provides for the use of an scFv of anti-CD18 antibodies to block HIV-1 transmission. Advantageously, such scFv (which is a single chain antibody in which the antigen-binding heavy and light chain regions are linked by a molecule that ensures that they fold in a manner that functionally resembles an Fab) is in a size range and of a structure (i.e., not being multi-chained) to be secreted by genetically engineered bacterial delivery systems such as, e.g., genetically engineered lactobacillus, genetically engineered E. coli, etc. Thus, bacteria (such as, e.g., lactobacillus, E. coli, etc.) that are genetically engineered to produce the scFv of a protective antibody (such as, e.g., anti-CD18 antibody) can be used to colonize a region (such as, e.g., the vaginal region, rectal region, etc.) and provide in situ protection against HIV infection.

The present invention provides methods of preventing the sexual transmission of HIV infection. The invention provides methods which utilize antibodies specific for CD18. CD18 is the β chain of two ICAM-1 integrin ligands: Mac-1 and LFA-1, both of which are present on migrating cells. The invention is based on a finding of being able to show that anti-CD18 can block transmission of cell-associated virus across an epithelial barrier and that, when applied to an epithelial surface, it can block infection by cell-free virus of susceptible cells below the epithelium. Without being bound by theory, explanatory factors for effectiveness of anti-CD18 against cell-free virus may be due to the virus acquiring host antigens when it buds from the cell it is infecting. The present invention also thereby addresses the problem previously observed with targeting viral antigens in the case of HIV, in which the antigens are highly variable and mutate frequently, for cell-free virus.

The invention in one preferred embodiment provides a method of preventing an initial HIV infection in a human or animal, comprising: exposing an epithelium (such as, e.g., a vaginal epithelium, cervical epithelium, rectal epithelium, colonic epithelium, oral epithelium) which may receive HIV (such as, e.g., cell-free HIV; cell-associated HIV) exposure to one or more anti-CD18 or anti-CD11 antibodies, or both, wherein the step of exposing the epithelium to the antibodies precedes any establishment of an HIV infection, and establishment of an HIV infection is prevented; such as, e.g., prevention methods including a step of exposing the epithelium to anti-ICAM antibodies in combination with the CD18 and/or CD11 antibodies; prevention methods including a step of delivering the antibodies to the epithelium via at least one bacterial (such as, e.g., lactobacillus, etc.) delivery system; prevention methods wherein an amount of bacterial (such as, e.g., lactobacillus, etc.) delivery system used is sufficient to express scFv, diabodies of scFv, triabodies of scFv, and/or tetramers of scFv-like molecules producible by bacteria in a concentration in a range of from 0.5 to 100 micrograms/ml; prevention methods wherein a concentration of scFv, diabodies of scFv, triabodies of scFv, and/or tetramers of scFv-like molecules ranges from about 0.5 to 100 micrograms/ml; prevention methods wherein a bacterial delivery system expresses scFv; prevention methods wherein the antibodies are expressed by a bacterial delivery system as a product (such as, e.g., scFv, diabodies of scFv, triabodies of scFv, tetramers of scFv-like molecules producible by bacteria, and combinations thereof); etc.

In another preferred embodiment, the invention provides a method of blocking transepithelial transmission of HIV (such as, e.g., cell-free HIV, cell-associated HIV) into a human or animal, comprising: exposing an epithelium (such as, e.g., a vaginal epithelium, cervical epithelium, rectal epithelium, colonic epithelium, oral epithelium) which may receive HIV (such as, e.g., cell-free HIV, cell-associated HIV) exposure to one or more anti-CD18 or anti-CD11 antibodies, or both; such as, e.g., blocking methods wherein the step of exposing the epithelium to the antibodies precedes any establishment of an HIV infection, and establishment of an HIV infection is prevented; blocking methods wherein transmission of HIV across the epithelium is reduced or prevented; blocking methods wherein sexual transmission of HIV is reduced or prevented; blocking methods including a step of exposing the epithelium to anti-ICAM antibodies in combination with the CD18 and/or CD11 antibodies; blocking methods including delivering the antibodies to the epithelium via at least one bacterial delivery system (such as, e.g., a lactobacillus delivery system, etc.); blocking methods wherein an amount of lactobacillus delivery system used is sufficient to express scFv, diabodies of scFv, triabodies of scFv, and/or tetramers of scFv-like molecules producible by bacteria in a concentration in a range of from 0.5 to 100 micrograms/ml; blocking methods wherein a bacterial delivery system expresses scFv; blocking methods wherein a concentration of scFv, diabodies of scFv, triabodies of scFv, and/or tetramers of scFv-like molecules ranges from about 0.5 to 100 micrograms/ml; blocking methods wherein the antibodies are expressed by a bacterial delivery system as a product (such as, e.g., scFv, diabodies of scFv, triabodies of scFv, tetramers of scFv-like molecules producible by bacteria, and combinations thereof); etc.

In another preferred embodiment, the invention provides a microbicide comprising: one or more anti-CD18 or anti-CD11 antibodies (with examples of anti-CD11 antibodies being anti-CD11a, anti-CD11b, anti-CD11c, anti-CD11d), or both, such as, e.g., microbicides wherein the antibodies are expressed by a bacterial delivery system as scFv (with examples being, e.g., microbicides wherein the antibodies are deliverable to a to-be-protected epithelium via at least one lactobacillus bacterial vehicle; etc.); microbicides further comprising at least one anti-ICAM antibody; microbicides wherein the cumulative anti-HIV effect of the anti-ICAM antibody or antibodies and the anti-CD18 and/or anti-CD11 antibodies together is at least equal to, or greater than, an additive effect of the anti-CD18 and/or anti-CD11 antibodies separately plus the anti-ICAM antibodies separately; etc.

The invention in another preferred embodiment provides a method of constructing a microbicide, comprising: (A) fusing an antigen-binding variable region of a light chain and an antigen-binding variable region of a heavy chain to a linker protein (such as, e.g., a linker protein comprising a sequence of amino acids wherein attached heavy and light chain variable regions fold into a configuration to which CD18 binds) to form a single chain antibody (scFv), wherein the antibody molecule may be the same or different and is selected from anti-CD11 and anti-CD18; (B) establishing conditions in which a bacterial delivery system (such as, e.g., lactobacillus delivery system, E. coli delivery system, lactococcus delivery system; etc.) expresses the single chain antibody of step (A) in an environment including an epithelium to be protected from HIV transmission.

In another preferred embodiment, the invention provides an expression product (such as, e.g., anti-CD18; anti-CD11; a ligand expressed by a recombinant bacteria; etc.) that blocks transepithelial transmission of HIV-1.

The methods, systems, products, compositions, etc. of the invention are useable and useful for females and males.

### BRIEF DESCRIPTION OF THE DRAWINGS

FIGS. 1A-1B are bar graphs showing that antibody to CD18 blocks migration of HIV-1 infected cells (FIG. 1A) and transmission of HIV-1 p24 (FIG. 1B) as well or better than antibody to ICAM-1. 1×10⁶ HIV-1 infected PBMC were added with designated antibodies (anti-ICAM-1 MT-M5, anti-CD18H52 or isotype control) at 100, 50 or 10 μg/ml to the apical side of HT-3 monolayers grown on permeable transwell supports, and allowed to transmigrate for 24 hours. Error bars represent +/−1 standard deviation in FIGS. 1A-5. For FIG. 1A, p<0.01 for each antibody treatment compared to untreated and isotype controls, and p<0.05 between anti-ICAM-1 and anti-CD18 groups at corresponding concentrations. For FIG. 1B, p<0.01 for each antibody treatment compared to untreated and isotype controls.

FIG. 2A is a bar graph and FIG. 2B is a line graph. A 50:50 mixture of anti-ICAM and anti-CD18 reduces migration of cells from HIV-1 infected cultures to a greater extent than either antibody alone. 1×10⁶ HIV-1 infected PBMC were added with designated antibodies (anti-ICAM-1 MT-M5, anti-CD18H52, or isotype control) or mixture at 50, 20 or 10 μg/ml to the apical side of HT-3 monolayers grown on permeable transwell supports, and allowed to transmigrate for 24 hours. FIG. 1A: p<0.05 for all antibody treatments compared to untreated and isotype controls. FIG. 1B: p<0.05 between each treatment at the same concentration.

FIG. 3 is a bar graph. Anti-CD18, used alone or in combination with anti-ICAM1, blocks transmission of HIV-1 p24 to a greater extent than anti-ICAM-1 alone. 1×10⁶ HIV-1 infected PBMC were added with designated antibodies (anti-ICAM-1 MT-M5, anti-CD18H52, or isotype control) or mix at 50, 20 or 10 μg/ml to the apical side of HT-3 monolayers grown on permeable transwell supports, and allowed to transmigrate for 24 hours. p<0.05 for all treatments compared to untreated and isotype controls; p<0.05 for anti-CD18 and 50:50 mix compared to anti-ICAM.

FIG. 4 is a bar graph. Blocking by anti-CD18 and enhancement by mixture with anti-ICAM-1 is not exclusive to one antibody clone. 1×10⁶ HIV-1 infected PBMC were added with designated antibodies (anti-ICAM-1 MT-M5, anti-CD18 7E4, or isotype control) or mix at 10 ug/ml to the apical side of HT-3 monolayers grown on permeable transwell supports, and allowed to transmigrate for 24 hours.

FIG. 5 is a bar graph. Anti-CD18 alone and in combination with anti-ICAM-1 reduces migration of cells from HIV-1 infected cultures. 1×10⁶ HIV-1 infected PBMC were added with designated antibodies (anti-ICAM-1 MT-M5, anti-CD18H52, or isotype control) or mix at 1 or 5 ug/ml to the apical side of HT-3 monolayers grown on permeable transwell supports, and allowed to transmigrate for 24 hours.

FIG. 6 is line graph (y=0.0073x−0.0473; R=0.9818).

FIG. 7 is a line graph, with data for anti-ICAM, anti-CD18 and 50/50 mix. FIG. 7A is a bar graph relating to FIG. 7.

FIG. 8 is a bar graph, for data from experimentation on untreated, isotype control, anti-ICAM, anti-CD18 and 50/50 mix.

FIGS. 9A-B are bar graphs. Anti-CD18 clone 7E4 blocks migration of HIV-1 infected cells (FIG. 9A) and transmission of HIV-1 p24 (FIG. 9B). 1×10⁶ HIV-1 infected PBMC were added with designated antibodies (anti-CD18H52, anti-CD18 7E4, or isotype control) at 50 or 10 μg/ml to the apical side of HT-3 monolayers grown on permeable transwell supports, and allowed to transmigrate for 24 hours. Data are expressed as mean±SD of basilar HIV-1 p24 concentration or viable PBMC counted, with three replicates per group. For both panels, p<0.05 for each antibody treatment compared to untreated and isotype controls.

FIGS. 10A-B are bar graphs. Anti-CD18 Fab block transmission of HIV-1-infected PBMC (FIG. 10A) and p24 (FIG. 10B) across an HT-3 cell monolayer. 1×10⁶ HIV-1 infected PBMC were added with designated treatment to apical side of HT-3 monolayers grown on permeable transwell supports and allowed to transmigrate for 24 hours. All intact antibodies were used at a concentrations of 50 or 10 μg/ml and all Fab were used at 34 or 6.7 μg/ml to equalize available binding sites. Data are expressed as mean±SD of viable PBMC counted (FIG. 10A) or basilar HIV-1 p24 concentration (FIG. 10B), and are representative of two separate experiments with three replicates per treatment group. For A, *p<0.05 for treatments compared to untreated and corresponding isotype control; for B, p<0.05 for all treatments compared to untreated and corresponding isotype controls.

FIGS. 11A-C are graphs. A 50:50 mixture of anti-ICAM and anti-CD18 reduces migration of cells from HIV-1 infected cultures (FIGS. 11A,B) and transmission of HIV-1 p24 to a greater extent than either antibody alone (FIG. 11C). 1×10⁶ HIV-1 infected PBMC were added with designated antibodies (anti-ICAM-1 MT-M5, anti-CD18 H52, or isotype control) or mixture at 50, 20 or 10 μg/ml to the apical side of HT-3 monolayers grown on permeable transwell supports, and allowed to transmigrate for 24 hours. Data are expressed as mean±SD of viable PBMC counted (FIGS. 11A,B) or basilar HIV-1 p24 concentration (FIG. 11C) and are representative of two separate experiments with three replicates per group. (FIG. 11A) p<0.05 for all antibody treatments compared to untreated and isotype controls. (FIG. 11B) p<0.05 between each treatment at the same concentration. (FIG. 11C) p<0.05 for all treatments compared to untreated and isotype controls; p<0.05 for anti-CD18 and 50:50 mixture compared to anti-ICAM.

FIGS. 12A-D are graphs. A 50:50 mixture of anti-ICAM and anti-CD18 7E4 reduces migration of cells from HIV-1 infected cultures (FIGS. 12A, B) and transmission of HIV-1 p24 (FIGS. 12C, D) to a greater extent than either antibody alone. 1×10⁶ HIV-1 infected PBMC were added with designated antibodies (anti-ICAM-1 MT-M5, anti-CD18 7E4, or isotype control) or mixture at a total amount of 20, 10 or 5 μg/ml to the apical side of HT-3 monolayers grown on permeable transwell supports, and allowed to transmigrate for 24 hours. Data are expressed as mean±SD of viable PBMC counted or basilar HIV-1 p24 concentration, and are representative of two separate experiments with three replicates per treatment group. (FIGS. 12A, C) p<0.05 for all antibody treatments compared to untreated and isotype controls. (FIG. 12B) p<0.05 between each treatment at the same concentration. (FIG. 12D) p<0.05 between mixture and other treatments at the 10 and 20 μg/ml concentration.

FIG. 13 is a graph showing data for experimentation using antibodies to ICAM-1 and CD18, both alone and in combination, to determine whether infection of target cells beneath the cervical epithelium is reduced. 20 ug/ml total of designated antibody or mixture was added to 1×10³ TCID50 HIV-1 JR-CSF immediately before addition to the apical side of an ME-180 transwell culture with 1×10⁶ PHA blasts in the basal compartment and incubated for 24 h at 37° C. Transwells were then removed, and PBMC supernatants were sampled at 48 h intervals. Infection of cells in the basilar compartment was measured by the level of increase of HIV-1 p24 in supernatants from the cultured cells. Data are expressed as mean of triplicate wells±SD, except for anti-CD18, which is a single well. Open symbols indicate a significant difference (p<0.05) from untreated cells. A combination effect is seen for anti-CD-18 and anti-ICAM-1, wherein most of the combination effect is considered attributable to anti-CD-18.

### DETAILED DESCRIPTION OF THE PREFERRED EMBODIMENTS OF THE INVENTION

In the present invention, protective antibodies (e.g., anti-CD18 antibodies, anti-CD11 antibodies (e.g., anti-CD11a antibodies; anti-CD11b antibodies; anti-CD11c antibodies; anti-CD11d antibodies), a combination of anti-CD18 and anti-CD11 antibodies) are used to interrupt transmission across an epithelium (such as, e.g., a vaginal epithelium, a cervical epithelium, a gastrointestinal epithelium, a rectal epithelium, a colonic epithelium, an oral epithelium) of pathogens (e.g., HIV).

For the protective antibodies mentioned herein, various iterations (such as, e.g., multi-chain antibodies, naturally occurring single chain antibodies, or fragments thereof, etc.) may be used in practicing the invention.

For delivering the protective antibodies of the present invention to an epithelium where they may be so used to interrupt transmission of a pathogen (such as HIV), a bacterial delivery system may be used, such as, e.g., a lactobacillus delivery system, an E. coli delivery system, etc. Other preferred bacterial systems to use include those based on bacterial species present as normal flora of the genital tract, gastrointestinal tract (such as, e.g., distal gastrointestinal tract), oral cavity, etc.

Use of lactobacilli is considered particularly preferred for constructing a delivery system. For example, a delivery system for an antibody can be provided using engineered lactobacilli for sustained in situ production of short surface-bound or secreted heterologous proteins known as single-chain Fvs (scFv). These scFv can fold to resemble the variable region of antibodies targeted toward specific HIV-related targets. It has been demonstrated that scFv against ICAM-1 can reduce in vitro transmission of cell-associated HIV-1 by 70±5%. Furthermore, in an in vivo transmission model using mice with severe combined immunodeficiency reconstituted with human peripheral blood mononuclear cells (Hu-PBL-SCID mice) antibody to ICAM-1 can provide up to 90% protection from transmission by HIV-1 infected cells inoculated intravaginally (Chancey, C. J., K. V. Khanna, J. F. Seegers, G. W. Zhang, J. Hildreth, A. Langan, and R. B. Markham, 2006, Lactobacilli-expressed single-chain variable fragment (scFv) specific for intercellular adhesion molecule 1 (ICAM-1) blocks cell-associated HIV-1 transmission across a cervical epithelial monolayer, J Immunol 176:5627-5636). These studies have indicated that this antibody is reacting with ICAM-1 on the surface of vaginal epithelial cells (data not shown). This observed in vivo protection was achieved with low concentrations of antibody (20 ug/ml). This lactobacillus delivery system is mentioned as an example and various other lactobacillus delivery systems may be constructed, as those in the art will appreciate. See, e.g., C. Kruger, Y. Hu, Q. Pan, H. Marcotte, A. Hultberg, D. Delwar, P. J. van Dalen, P. H. Pouwels, R. J. Leer, C. Kelly, C. Van Dollenweerd, J. Ma, and L. Hammartstrom, “In situ delivery of passive immunity by lactobacilli producing single-chain antibodies,” Nature Biotechnology, 20:702-706 (2002).

For in situ use of lactobacilli which can constitutively produce antibody over a sustained period, adequate concentrations of antibody are needed to interrupt transmission of the pathogen (e.g., HIV). Such antibody concentrations for in situ use can be provided when using anti-CD18 and/or anti-CD11 antibodies, and optionally combined with use of anti-ICAM antibodies, to provide anti-transmission effect.

The antibodies expressed by a bacterial system (e.g., lactobacilli, E. coli) are not “traditional antibodies” but are of the category referred to as single chain antibodies (scFv) generated by genetically fusing the functional end of the light and heavy chains of an antibody molecule (e.g., anti-CD18 antibody; anti-CD11 antibody) to a linker protein. Use of linker proteins is known to those in the art, see, e.g., Tang, Y., N. Jiang, C. Parakh, and D. Hilvert, 1996, Selection of linkers for a catalytic single-chain antibody using phage display technology, J Biol Chem, 271:15682-15686. Additionally, in practicing the invention there may be used variants and alternatives, such as, e.g., diabodies, tribodies, and scFv tetramers which are variants of scFv that include more binding sites. See, e.g., Le Gall, F., S. M. Kipriyanov, G. Moldenhauer, and M. Little, 1999, Di-, tri- and tetrameric single chain Fv antibody fragments against human CD19: effect of valency on cell binding, FEBS Lett 453:164-168; Lawrence, L. J., A. A. Kortt, P. Iliades, P. A. Tulloch, and P. J. Hudson, 1998, Orientation of antigen binding sites in dimeric and trimeric single chain Fv antibody fragments, FEBS Lett 425:479-484. As the linker protein also may be used any sequence of amino acids that ensure that the attached heavy and light chain variable regions fold in a manner that the antigen of interest (e.g., CD18) binds.

The bacterial delivery system may be selected according to the application. For example, using a lactobacillus bacterial delivery system is considered preferable for formulating a vaginally applicable microbicide. By way of another example, using an E. coli delivery system is considered preferable for formulating a rectally applicable product (such as, e.g., a product to prevent transmission via rectal intercourse).

For practicing the present invention, the active ingredients may be in various forms, such as, e.g., a microbicide, a delayed release delivery system, a solid phase structure, cervical rings, sponges, condoms, gels, creams, suppositories, capsules, etc. A microbicide is a most preferred formulation for the inventive active ingredients, but it should be appreciated that microbicide formulations may be varied. For example, the protective antibodies may be delivered by systems incorporating a delivery vehicle (such as, e.g., a delayed release delivery system) or in a solid phase structure from which the protective antibodies may be slowly released (such as, e.g., solid phase materials impregnated with protective antibody or fragments; cervical rings; etc.). Alternately, the protective antibodies may be produced in plants. Additionally, as purification technologies improve for practical use, purified protective antibodies may be prepared and administered directly to a human or animal, without a bacteria delivery system, such as administration by gels, cervical rings, sponges, etc.

Administration of the active ingredients may be, preferably, by the consumer himself or herself, such as, e.g., bacteria expressing the scFv being delivered in lyophilized form (such as in, e.g., a suppository, a capsule, etc.) via self-administration by the consumer. Studies elsewhere have shown certain bacteria to remain viable, without refrigeration, for up to two years in suppository or capsule form; similar viability for bacterial systems according to the present invention may be expected. It will be appreciated that bacteria are not expected to survive forever and therefore replacement may be in order, such as, e.g., every several days or monthly. The invention also provides for the bacteria to also encode a protein useable as part of a detection system to monitor persistence of the bacteria, as an indicator of when a next application of bacteria may be needed.

It should be appreciated that female or male examples may be given herein, by way of example and not to limit the invention thereto. The inventive methods, products and formulations are useful in protecting men and women from transepithelial HIV transmission (such as transepithelial HIV transmission otherwise resulting from sexual intercourse).

Advantageously, in the present invention, bacteria may be engineered to produce the protective antibodies, after which production of the product simply involves growing the bacteria in large quantities and then lyophilizing the bacteria for in situ delivery, which can be done relatively inexpensively. Thus, the invention advantageously provides methods of preventing initial HIV infection and preventing transepithelial HIV transmission in which no purification of antibody is necessary and no complex chemical processes are required to synthesize an active product.

### EXAMPLES

### Example 1

In this Example we have identified antibody to CD18, which is one chain for the LFA-1 or Mac-1 molecules that can serve as ligands for ICAM-1, as a candidate for disruption of cell-associated HIV-1 transmission across the cervical epithelium. This Example also demonstrates that antibody to CD18 used in combination with antibody to ICAM-1 reduces migration of HIV-1 infected cells to a greater degree than either antibody alone, providing effective blockade of transmission at antibody concentrations that should be obtainable in a clinical setting.

### Materials and Methods

### HIV-1 Preparation

HIV-1Ba-L (Advanced Biotechnologies, Inc., Columbia, Md.), a CCR5-utilizing variant of HIV-1, was purchased in 1.0 ml aliquots (1×10⁶ 50% tissue culture infectious doses (TCID₅₀)/ml) and stored in liquid nitrogen.

### PBMC Isolation and Culture

Human PBMC were isolated by centrifugation of leukopheresed blood (Hemapheresis Center, Johns Hopkins Hospital, Baltimore, Md.) on a Ficoll Plus gradient (GE Healthcare, Piscataway, N.J.). PBMC were cultured at 2×10⁶ cells/ml for 48 hours in RPMI-1640 supplemented with 100 U/ml penicillin/100 μg streptomycin, 10 ng/ml gentamicin, and 2 mM L-glutamine (hereafter referred to as cRPMI; media and all supplements from Mediatech, Herndon, Va.), 10% heat-inactivated fetal calf serum (FCS; Atlanta Biologicals, Atlanta, Ga.), and 5 μg/ml PHA (Sigma, St. Louis, Mo.) at 37° C., 5% CO₂. After 48 hours, PBMC were transferred to cRPMI-10% FCS supplemented with 10 U/ml IL-2 (Roche Biochemicals, Indianapolis, Ind.) and incubated with 10⁴ TCID₅₀ HIV_{BtL} for 24 hours. The virus inoculum was removed after 24 hours, cells were washed once with warm cRPMI, and fresh cRPMI-10% FCS supplemented with 10 U/ml IL-2 was added. Cultures were fed with fresh media on days 3 and 7 post-infection (pi). PBMC were used for transmission experiments on days 7-9 post infection.

### Human Cervical Epithelial Cell Transwell Cultures

The human, spontaneously-transformed, cervical epithelial cell line HT-3 (American Type Cell Culture, Rockville, Md., ATCC# HTB-32), was cultured in McCoy's 5a medium supplemented as described above for cRPMI, with 10% FCS, and routinely sub-cultured every 3 days with cell displacement by 0.05% trypsin-EDTA (Mediatech). Cervical epithelial cells were plated at 2×10⁵ cells in cRPMI-10% FCS per 12 mm diameter PCF transwell insert with pore size of 3.0 μm (Millipore, Bedford, Mass.) and were maintained at 37° C., 5% CO₂ conditions. Media were changed every 2-3 days. The cells formed a polarized, complete monolayer on the transwell inserts in 7 days, which was confirmed by monitoring permeability of cell monolayers to horseradish peroxidase (Sigma). The cervical epithelial monolayers on transwell inserts were used between days 6 and 8 of culture.

### Transepithelial HIV-1 Transmission

The transepithelial migration assay was performed as described by Bomsel (Bomsel, M., 1997, Transcytosis of infectious human immunodeficiency virus across a tight human epithelial cell line barrier, Nat Med 3:42-7), with several modifications. Briefly, 1×10⁶ HIV_{Ba-L}-infected PBMC were added to the apical sides of cervical epithelial monolayers with antibody treatments as designated and allowed to transmigrate for 24 hours. Viability of PBMC was assessed by trypan blue exclusion (Sigma) prior to their addition to the transwells and was always found to be >85%. The amount of HIV-1 p24 antigen in the apical and basal supernatant fluid was determined by HIV-1 p24 ELISA assay (Perkin-Elmer, Boston, Mass.). Transmitted PBMC were collected from the basal compartment, pelleted by centrifugation, and counted on a hemacytometer with trypan blue exclusion to assess viability. Mouse monoclonal antibodies H52 (anti-CD18) and MT-M5 (anti-ICAM-1) were obtained from the laboratory of James Hildreth, Johns Hopkins University School of Medicine. Mouse monoclonal antibody 7E4 (anti-CD18) was obtained from Beckman-Coulter Immunotech (Miami, Fla.). Mouse myeloma IgG1 isotype control was obtained from Zymed (South San Francisco, Calif.).

### Statistical Analysis

Statistical analysis was performed using the Intercooled Stata 8 (Stata Corp, College Station, Tex.) statistical package. One-way ANOVA with Bonferroni correction was used to compare the differences between groups, and p values equal to or less than 0.05 were considered significant.

### Experimental Results

### Antibody to CD18 Blocks Transmission of Cell-Associated HIV-1 Across a Cervical Epithelial Monolayer

In order to compare the relative efficacy of antibody to CD18 in blocking cell-associated HIV-1 transmission to that of anti-ICAM-1, either anti-ICAM-1 (clone MT-M5), anti-CD18 (clone H52), or isotype control mouse IgG1 was added to 1×10⁶ HIV-infected PBMC immediately prior to their addition to the apical chamber of cervical epithelial transwell cultures. PBMC were allowed to migrate for 24 hours and antibodies remained present for the duration of the assay.

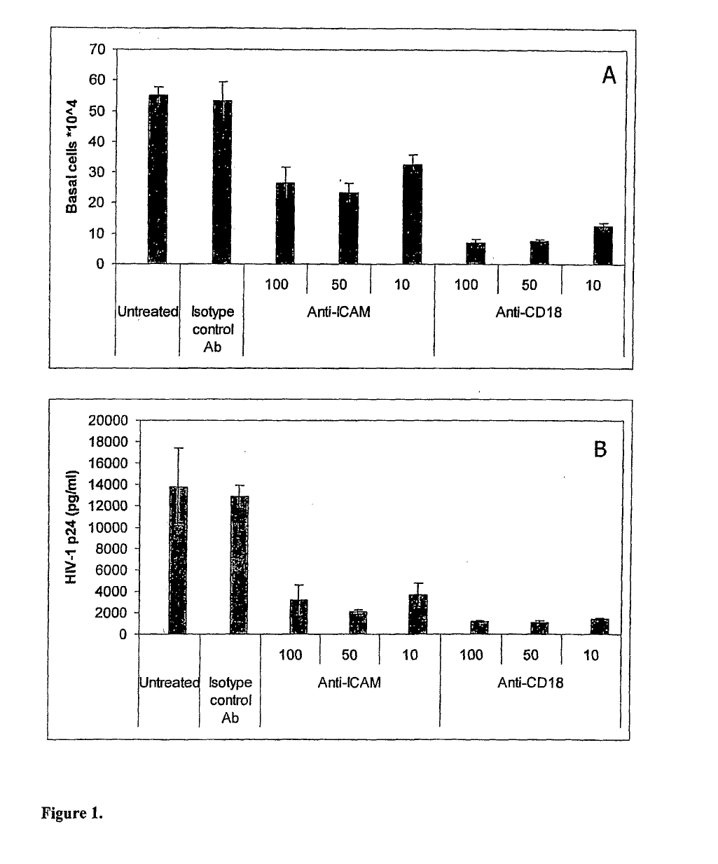

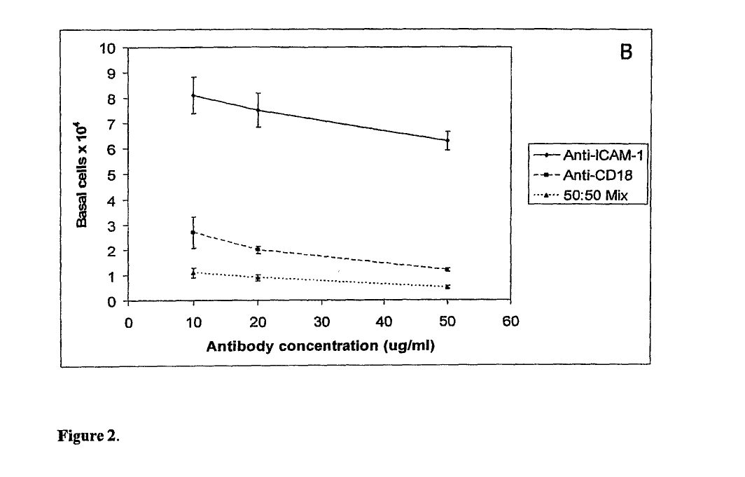

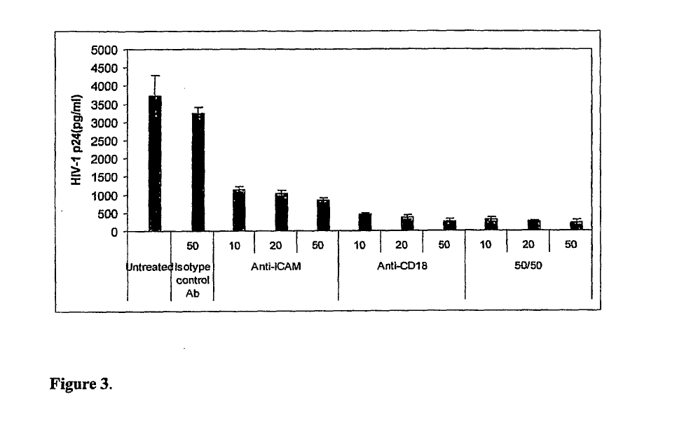

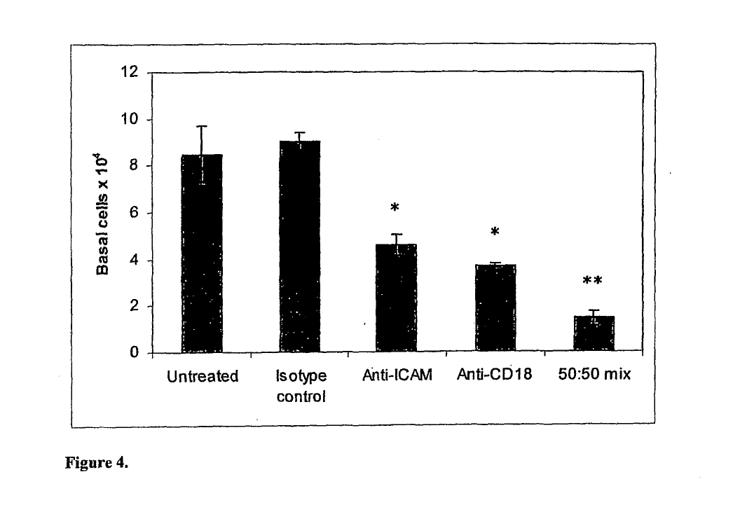

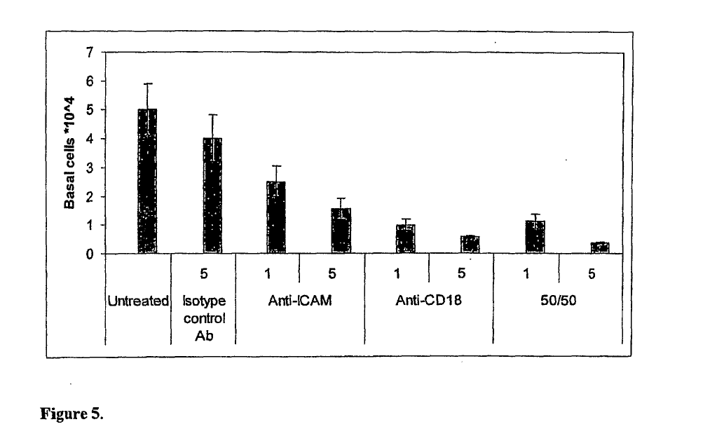

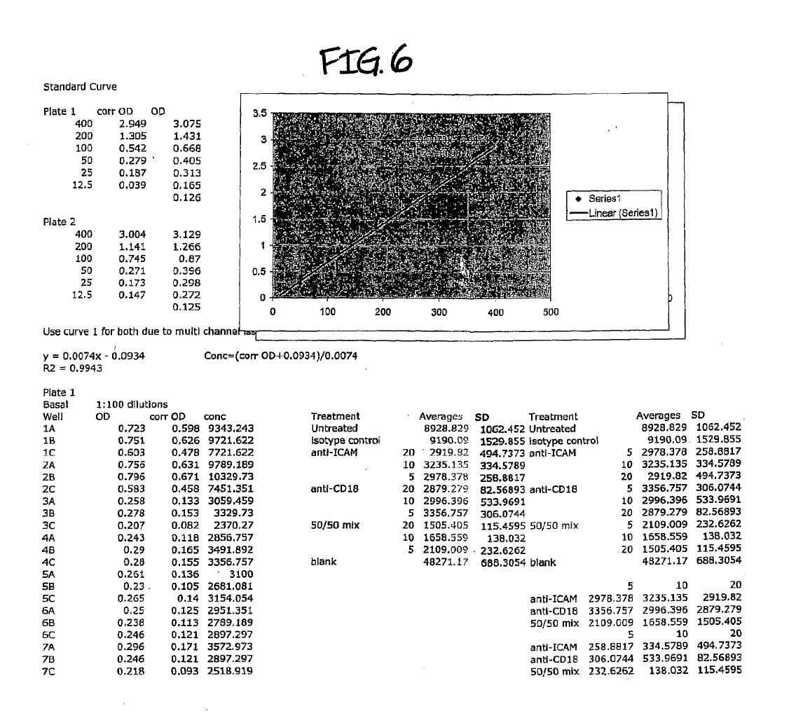

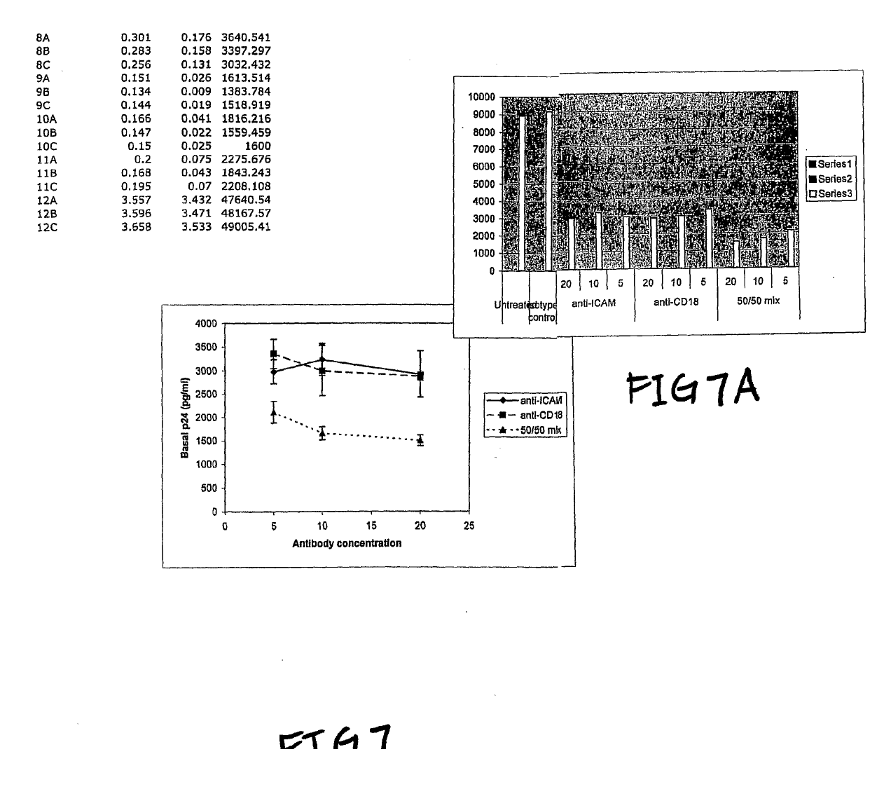

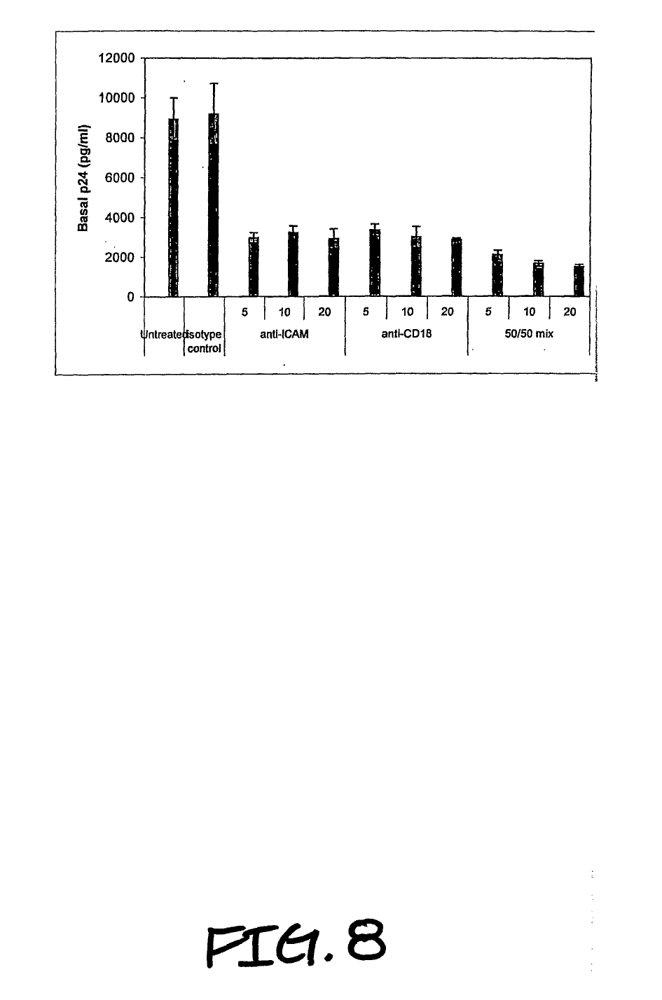

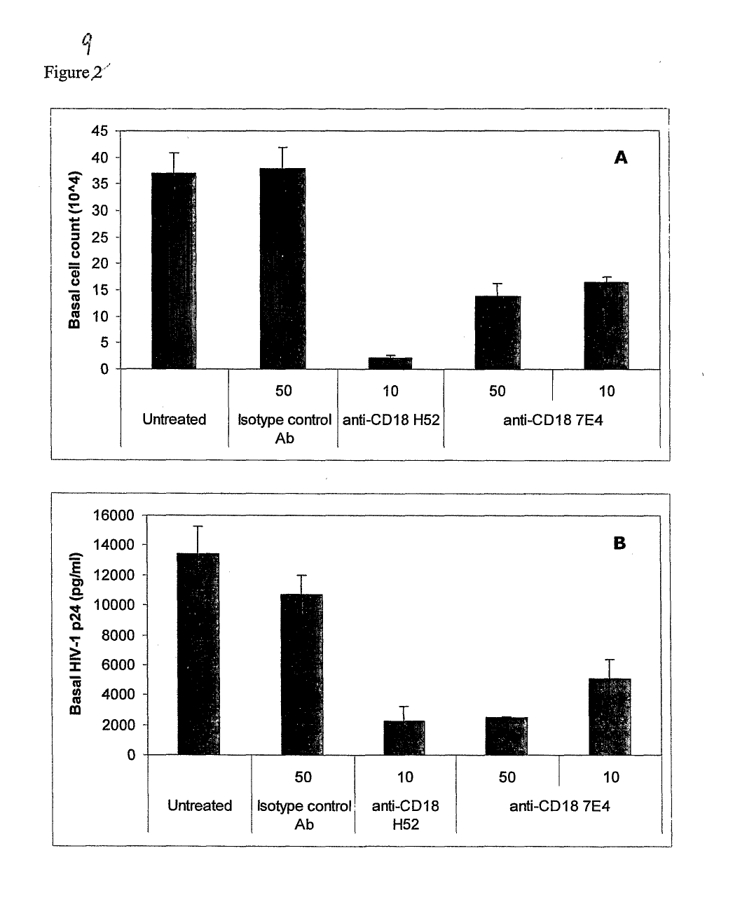

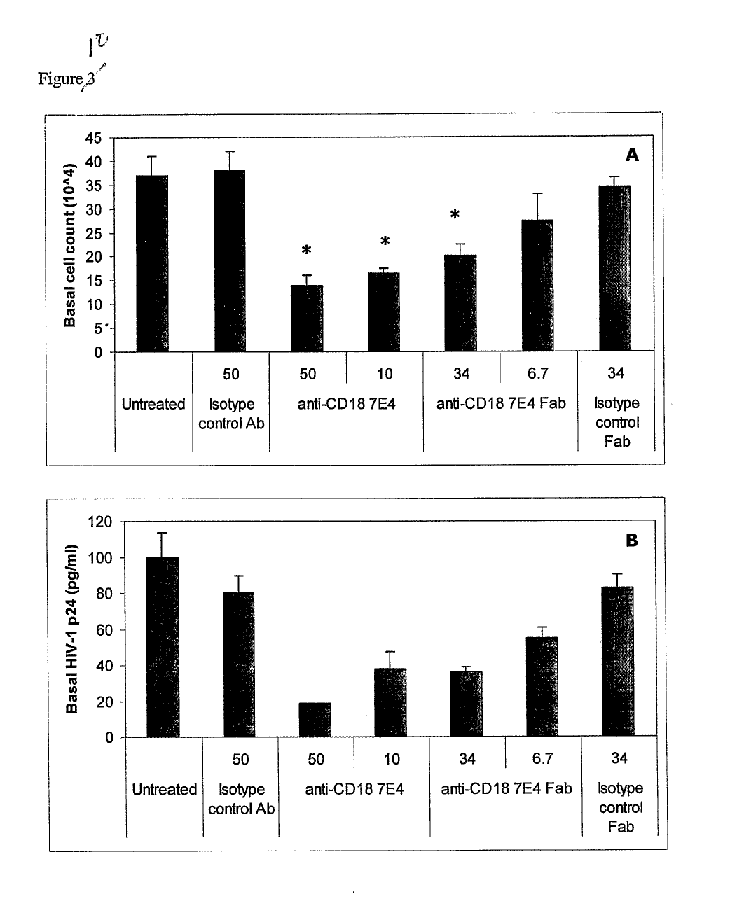

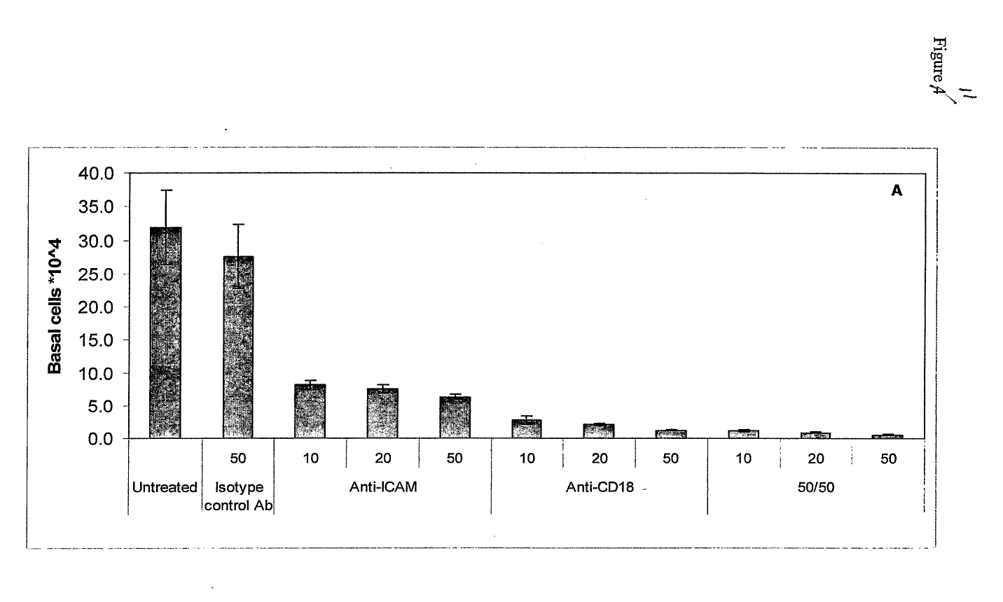

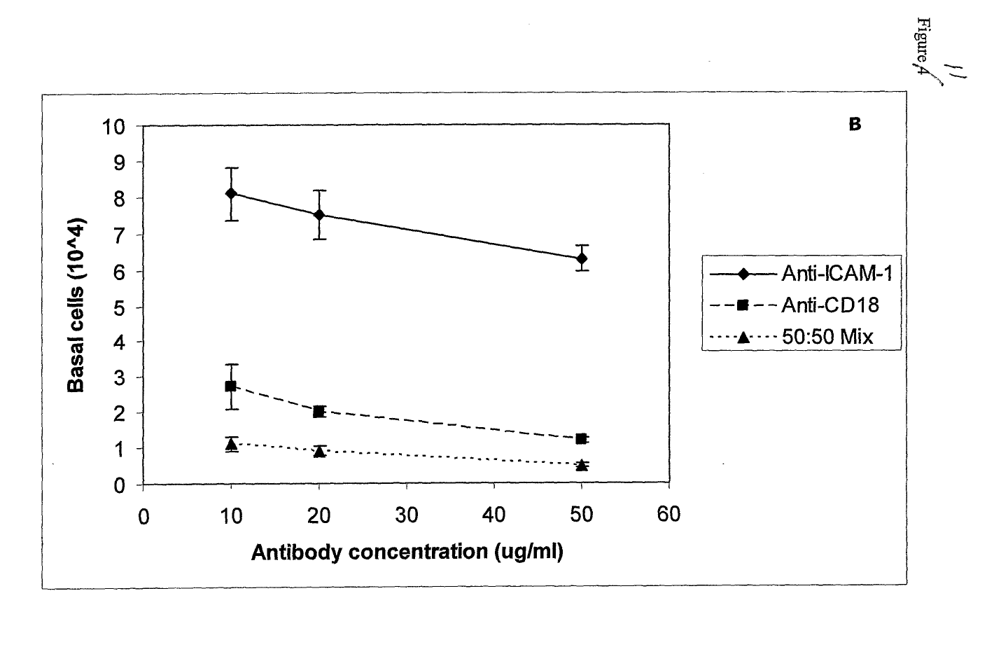

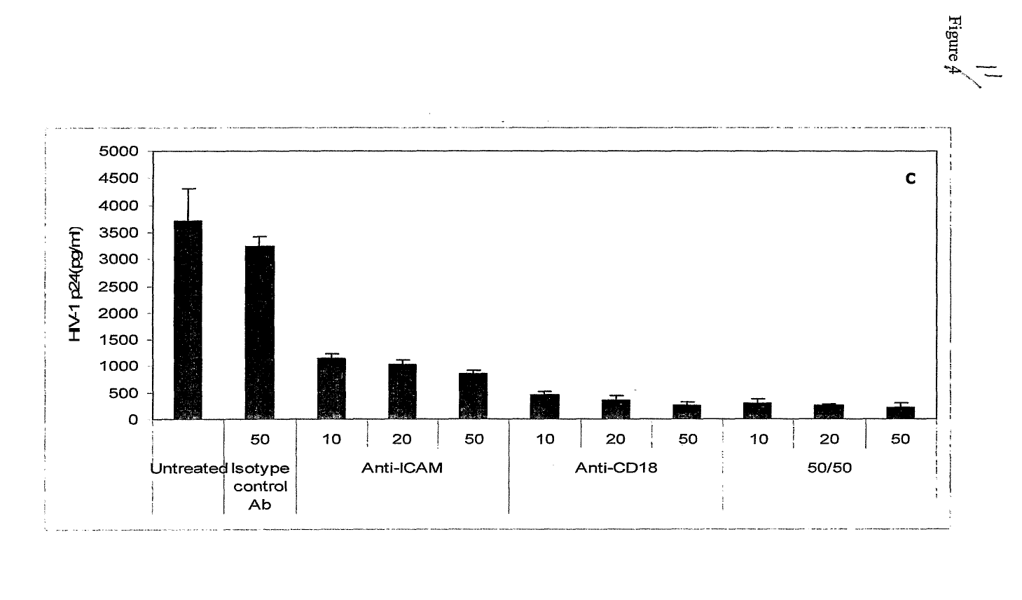

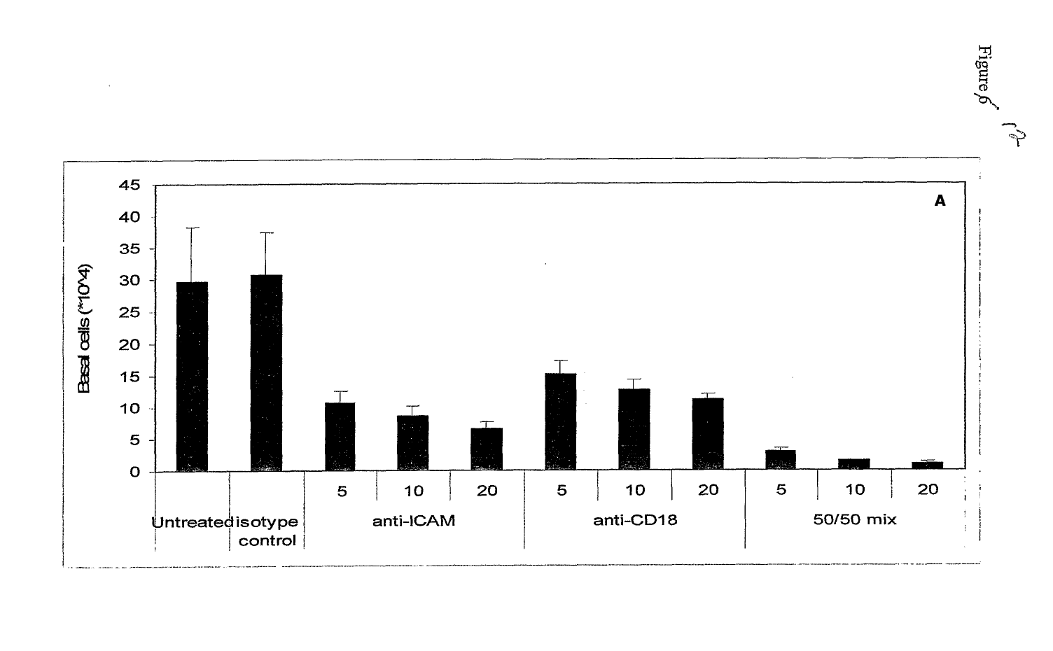

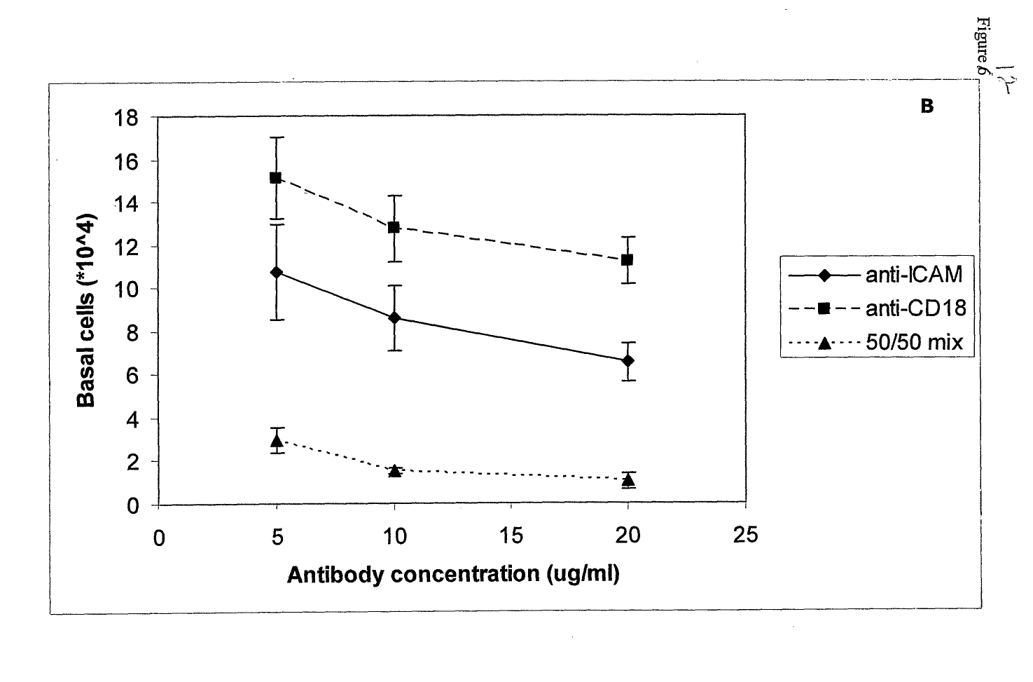

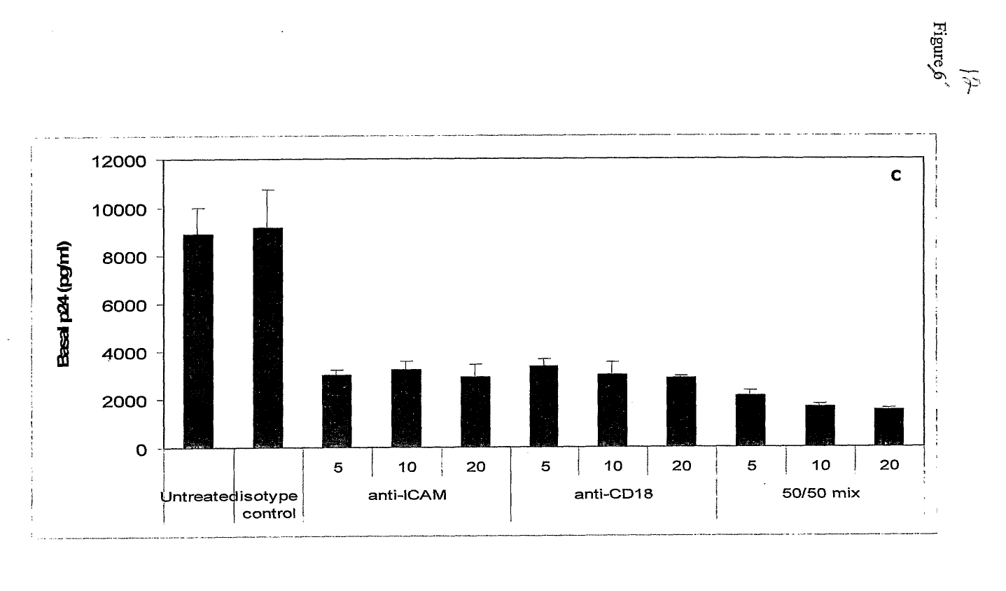

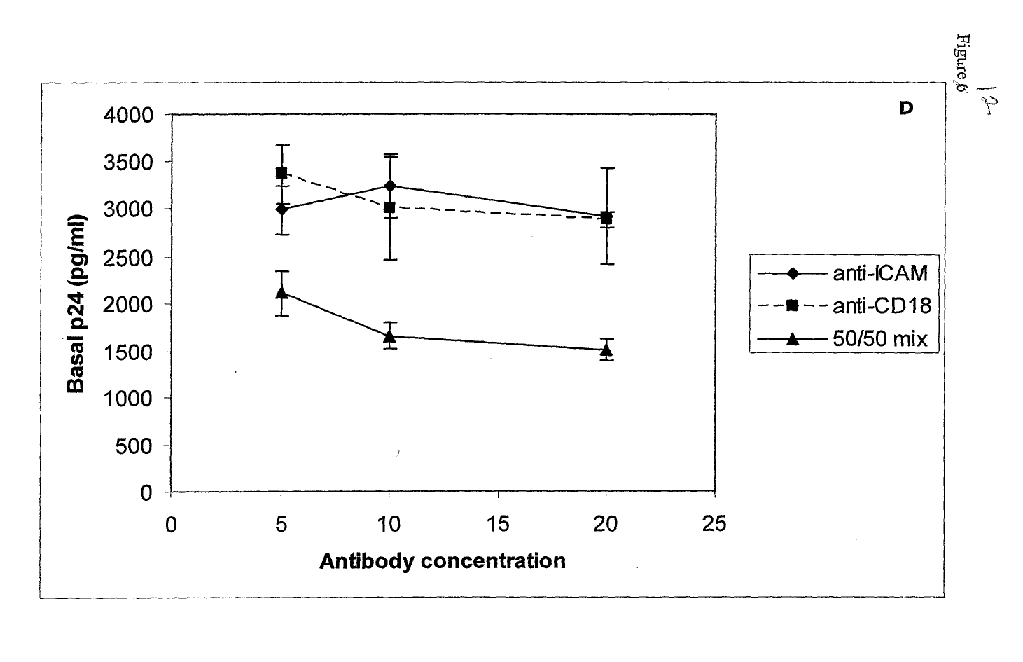

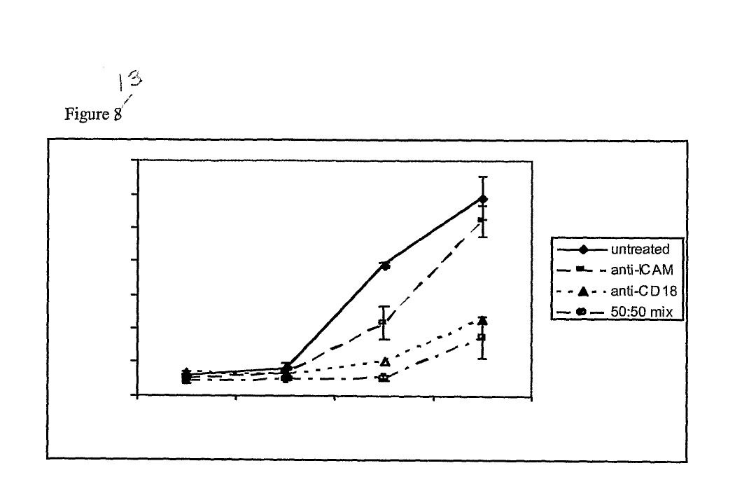

Both anti-ICAM-1 and anti-CD18 significantly (p<0.01) reduced cell migration at all concentrations tested over a range of 10-100 μg/ml (FIG. 1A) when compared to both untreated and isotype controls. However, anti-CD18 blocked cell migration significantly better (p<0.05) than anti-ICAM-1 at all concentrations tested, further reducing the number of cells detected in the basal compartment by 2.5- to 4-fold when compared to blocking by the corresponding concentration of anti-ICAM-1. A similar pattern was observed by measuring the amount of HIV-1 p24 detected in the basal side supernatant. Both antibody treatments at each concentration significantly (p<0.01) reduced the amount of HIV-1 p24 detected in the basal compartment (FIG. 1B). Less HIV-1 p24 was detected in the anti-CD18 treatment groups than in the corresponding anti-ICAM-1 treatment groups; however, the difference was not statistically significant. Treatment with antibody did not alter the amount of HIV-1 p24 released by cells on the apical side of the transwells (data not shown).

The levels of blocking observed in vitro for anti-CAM-1 have previously correlated to a highly significant reduction in cell-associated HIV-1 transmission using the in vivo HUPBL-SCID mouse model (Chancey et al., supra.). Therefore, the high degree of blocking observed in these experiments demonstrate that antibody to CD18 has significant potential for development as an anti-HIV microbicide.

50:50 Mix of Anti-CAM-1 and Anti-CD18 Antibodies Reduces Migration of Cells from HIV-1 Infected Cultures

In order to determine whether a mix of antibodies to different adhesion molecules could block to a higher degree than a single antibody, antibodies to CD18 and ICAM-1 mixed at a 50:50 ratio were added to 1×10⁶ PBMC immediately prior to their addition to the apical chamber of cervical epithelial transwell cultures. HIV-1 infected PBMC were allowed to migrate for 24 hours and antibodies remained present for the duration of the assay.

All antibody treatments at all concentrations reduced transmigration of PBMC from infected cultures significantly (p<0.05) when compared to untreated or isotype-control treated transwells (FIG. 2A). Anti-CD18 again reduced cell migration to a greater extent than anti-ICAM-1, and treatment with the anti-ICAM-1/anti-CD18 50:50 mix yielded a statistically significant reduction (p<0.05) in cell migration beyond that observed at corresponding concentrations with either anti-ICAM-1 or anti-CD18 alone (FIG. 2B).

When the concentration of antibody was reduced further, enhancement of blocking of cell migration with a 50:50 mix was observed at a concentration of 5 ug/ml, but at 1 ug/ml the 50:50 mix blocked no better than anti-CD18 alone (FIG. 5). Because anti-ICAM-1 at 1 ug/ml only reduced transmission by 38% and was only marginally statistically significant (p=0.051), this may represent a loss of the contribution of the anti-ICAM-1 at low concentration.

This enhancement of blocking of migration of HIV-1 infected cells by a 50:50 mix of antibodies to the adhesion receptor pair anti-ICAM-1 and anti-CD18 is notable because it suggests that a more pronounced effect may be achieved with a lower total amount of each antibody. Increased efficacy at lower concentrations would be desirable in an antibody-based microbicide regardless of delivery system. The data on transmission of infected cells makes clear that inhibition can be established at very low concentrations of each antibody. Therefore, antibody to CD18 alone and in combination with antibody to ICAM-1 should offer a method to block cell-associated HIV-1 transmission and this can be achieved with antibody concentrations which can realistically be expected to be achievable in vivo as a result of in situ production by transformed bacteria.

### Example 2

### Antibodies and Fab Generation

Mouse IgG1 anti-human monoclonal antibodies H52 (anti-CD18) and MT-M5 (anti-ICAM-1) were obtained from the laboratory of James Hildreth, Johns Hopkins University School of Medicine. Mouse IgG1 anti-human monoclonal antibody 7E4 (anti-CD18) was obtained from Beckman-Coulter Immunotech, Miami, Fla. Mouse myeloma IgG1 isotype control was obtained from Zymed, South San Francisco, Calif. Hamster anti-mouse ICAM-1 and hamster IgG1 isotype control were purchased from BD Biosciences/Pharmingen (San Diego, Calif.). For Fab studies, anti-CD18 clone 7E4 and mouse IgG1 isotype control was digested and purified using a ficin-based Immunopure Fab/F(ab)′2 digestion kit (Pierce, Rockford Ill.).

HuPBL-SCID mouse model of vaginal transmission The HuPBL-SCID mouse model was previously described (Khanna, K. V., K. J. Whaley, L. Zeitlin, T. R. Moench, K. Mehrazar, R. A. Cone, Z. Liao, J. E. Hildreth, T. E. Hoen, L. Shultz, and R. B. Markham, 2002, Vaginal transmission of cell-associated HIV-1 in the mouse is blocked by a topical, membrane-modifying agent, J Clin Invest 109:205-211). Briefly, female mice with severe combined immunodeficiency (C.B-17 SCID) (Bosma, G. C., R. P. Custer, and M. J. Bosma 1983, A severe combined immunodeficiency mutation in the mouse, Nature 301:527-530), were obtained from a SCID mouse colony (established using C.B-17 SCID mice from Jackson Laboratories, Bar Harbor, Me.). The mice were administered 5×10⁷ unstimulated, uninfected HuPBMC intraperitoneally (i.p.) in 1 ml PBS, followed 7 days later by treatment subcutaneously (s.c.) with 2.5 mg progestin (Depo-Provera®, Upjohn Pharmaceuticals, Kalamazoo, Mich.). Seven days following progestin treatment, the mice were anesthetized with isoflurane (IsoFlo, Abbott Laboratories, Chicago Ill.) in a 30-50% O₂ mix delivered by a Vaporstick anesthesia apparatus (SurgiVet Inc., Waukesha, Wis.) and intravaginally administered either a total of 0.4 μg in 10 μl of anti-ICAM-1 (MT-M5), anti-CD18 (H52), or a 50:50 anti-ICAM-1:anti-CD18 mix as designated or the appropriate mix of isotype control antibodies 5 minutes prior to receiving 1×10⁶ HIV-1_{Ba-L}-infected HuPBMC suspended in PBS-1% bovine serum albumin (BSA, Sigma). Mice remained anesthetized for 5 minutes following intravaginal inoculation by pipette, and no leakage of inocula was observed. Extreme care was taken to avoid trauma to vaginal tissues. Two weeks later the mice were euthanized and peritoneal cells were recovered by lavage with cold PBS. The cells recovered by lavage (of both murine and human origin) were assayed by DNA-PCR for human β-globin to confirm the success of the human cell engraftment in the mice. Mice without human cells, typically between 0 and 30% of an experimental group, were excluded from analyses.

To assess virus recovery from cells harvested from the peritoneal cavities of challenged mice, uninfected HuPBMC were stimulated with PHA and maintained in IL-2 supplemented media (1×10⁶ per mouse) in preparation for co-culture with the peritoneal cells recovered from the HuPBL-SCID mice. Positive mice were determined by HIV-1 p24 ELISA on supernatants from co-cultured cells, which in our experience has been the most sensitive method for detecting infected mice. In all cases the cells were obtained from a donor other than that from whom cells were obtained for the original transplant into the peritoneal cavities of the mice.

HuPBMC that were used for vaginal inoculation were isolated as described above and maintained in cRPMI-1640. PBMC were stimulated with PHA for 2 days; cells were then exposed to 10⁴ TCID₅₀ of HIV-1_{Ba-L} in cRPMI with IL-2 (10 U/ml). Infected-cell cultures were maintained in cRPMI supplemented with IL-2 for 10 days prior to inoculation into the mice.

### Example 3

### Antibody to CD18 Blocks Transmission of Cell-Associated HIV-1 Across a Cervical Epithelial Monolayer

Reduction of cell migration (as discussed above in Example 1) was not restricted to anti-CD18 derived from a single hybridoma; when a second blocking antibody to CD18, clone 7E4, was tested at 50 and 10 μg/ml, migration of cells from infected cultures was reduced by 63% and 55%, respectively (FIG. 9A). A larger reduction of transmission was observed using basal HIV-1 p24 as an indicator of transmission, with 7E4 at 50 and 10 μg/ml reducing transmission of p24 by 81% and 62%, respectively (FIG. 9B). Blocking measured by both criteria was highly statistically significant (p<0.01), although of a lesser magnitude than the reduction of migration and transmission observed with anti-CD18 clone H52.

### Example 4

### Anti-CD18 Fab Block Cell-Associated HIV-1 Transmission In Vitro

Fab fragments of anti-CD18 clone 7E4 were tested for their ability to block HIV-1 p24 transmission and migration of infected cells in vitro, in order to determine whether monovalent antibody fragments with the same number of antigen binding sites per molecule and lacking the Fc region of the antibody molecule would block as well as intact antibody. Intact anti-CD18, isotype control antibody, anti-CD18 Fab, or isotype control Fab were added along with HIV-1 infected PBMC to the apical sides of HT-3 cervical epithelial monolayers and the cells were allowed to migrate for 24 hours. Concentrations of Fab were adjusted to equalize the number of anti-CD18 binding sites.

At a concentration of 34 μg/ml, the anti-CD18 Fab significantly (p<0.05) reduced both migration of PBMC from HIV-1 infected cultures and transmission of HIV-1 p24 (FIG. 10A-B) compared to both untreated and isotype controls. When the concentration was reduced to 6.7 μg/ml, transmission of HIV-1 p24 was significantly reduced (FIG. 10B) though cell migration showed a statistically insignificant reduction compared to isotype control Fab (FIG. 10A). However, at both concentrations tested, the anti-CD18 Fab blocked transmission less efficiently than the corresponding concentration of intact anti-CD18. At 34 μg/ml Fab and 50 g/ml intact anti-CD18, transmission was reduced by 45.5±6.8% and 62.5±6.3% respectively for cell migration and 63.8±2.6% and 71.3±0.2% respectively for p24.

### Example 5

### 50:50 Mixture of Anti-ICAM-1 and Anti-CD18 Antibodies Reduces Transmission of Cell-Associated HIV-1

In order to determine whether a mixture of antibodies to each member of the receptor-ligand pair involved in the integrin-adhesion molecule interaction could block more efficiently than the same total concentration of a single antibody, antibodies to CD18 and ICAM-1 mixed at a 50:50 ratio were added to 1×10⁶ PBMC immediately prior to their addition to the apical chamber of cervical epithelial transwell cultures. PBMC were allowed to migrate for 24 hours and antibodies remained present for the duration of the assay.

Anti-ICAM-1 and anti-CD18 treatments at all concentrations tested reduced transmigration of PBMC from infected cultures significantly (p<0.05) when compared to untreated or isotype-control treated cultures (FIG. 11A). Anti-CD18 again reduced cell migration to a greater extent than anti-ICAM-1, and treatment with the anti-ICAM-1:anti-CD18 50:50 mixture yielded a small but statistically significant reduction (p<0.05) in cell migration beyond that observed at corresponding concentrations with either anti-ICAM-1 or anti-CD18 clone H52 alone (FIG. 11B). Though a significant reduction in transmitted HIV-1 p24 could be observed when comparing anti-CD18 and the 50:50 mixture to corresponding concentrations of anti-ICAM-1, there was only a slight and statistically insignificant reduction observed when comparing the anti-ICAM/anti-CD18 mixture with anti-CD18 used alone (FIG. 11C). When the concentration of antibody was reduced further, enhancement of blocking of cell migration with a 50:50 mixture was observed at a concentration of 5 μg/ml (FIG. 5).

### Example 6

### Anti-CD18 Clone 7E4 with Anti-ICAM-1

A dramatic effect was observed when mixing anti-CD18 clone 7E4, which blocked less efficiently than H52 when used alone, with anti-ICAM-1. At all concentrations tested, a 50:50 mixture of anti-ICAM-1 and anti-CD18 7E4 reduced migration of HIV-1 infected cells significantly more than corresponding concentrations of either antibody alone (FIGS. 12A-B). A similar effect was observed using HIV-1 p24 as the indicator, with the 50:50 mixture reducing transmission significantly more than corresponding antibodies alone at the 10 μg/ml and 20 μg/ml concentrations (FIGS. 12C-D). Most notably, the 5 μg/ml concentration of the 50:50 mixture reduced cell migration by 90±2.0% compared to untreated samples, a significantly greater reduction than either anti-ICAM-1 or anti-CD18 7E4 at 20 μg/ml (78±3.6 and 62±3.0% respectively (FIG. 12B)).

### Example 7

### Anti-ICAM, Anti-CD18, and 50:50 Mixture Block Transmission of Cell-Associated HIV-1 In Vivo

Antibody to ICAM-1 on the cervical epithelium has been observed to reduce transmission of cell-associated HIV-1 in an in vivo model utilizing Depo-Provera-treated HuPBL-SCID mice. In order to assess whether blocking CD18 on migrating cells alone or in combination with blocking ICAM-1 on the murine vaginal epithelium could block HIV-1 transmission in vivo in the same model, either anti-mouse ICAM-1, anti-CD18, a 50:50 mixture of anti-mouse ICAM-1 and anti-CD18, or a mixture of isotype control antibodies (0.4 μg of single antibodies or 0.2 μg of each antibody for mixtures in 10 μl PBS-1% BSA) were administered intravaginally, followed five minutes later by intravaginal inoculation of 1×10⁶ HIV-1 infected human PBMC. Anti-mouse ICAM-1, anti-CD18, and the anti-ICAM: anti-CD18 mixture all significantly reduced transmission of cell-associated HIV-1 compared to the control group (Table 1). The concentration of antibodies used yielded complete protection in the groups treated with a single antibody.

TABLE 1
Antibodies to ICAM-1 and CD18 reduce transmission
of cell-associated HIV-1 to HuPBL-SCID mice.

| Treatment | HIV-positive mice/total |
|---|---|
| Anti-mouse-ICAM-1 | 0/8 (0%), p < 0.01 |
| Anti-human-CD18 | 0/8 (0%), p < 0.01 |
| Anti-mouse-ICAM-1 + anti-human-CD18 | 2/6 (33%), p = 0.053, * |
| Isotype control | 6/7 (86%) * |

* 1-2 mice excluded for HuPBMC engraftment failure.

### Example 8

### Anti-CD18 Applied to Epithelium Prevents Free-Virus Initiated Infection of Susceptible Sub-Epithelial Cells

Because the relative importance of cell-free and cell-associated virus in sexual transmission is unknown, the efficacy of anti-CD18 and anti-ICAM-1 in transmission of cell-free virus was evaluated. Cell-free virus cannot be transmitted in the Hu-PBL-SCID mouse system, so the efficacy of this approach was evaluated in the in vitro transwell model, using human serum. An investigation was made of whether antibodies added to the upper chamber could reduce infection by free virus of susceptible PBMC (PHA and IL-2 activated) that were placed in the lower chamber prior to inoculation of the upper chamber with cell-free virus and different concentrations of the Mabs. After 24 hours, the transwells, cells were removed from the lower chamber, washed, and re-suspended in IL-2 containing culture media. The concentration of p24 in the culture supernatant fluid was determined over 7 days. The data in FIG. 13 indicate that the concentration of p24 was significantly lower in the transwells to which anti-CD18 had been added to the upper chamber. Also, the anti-ICAM-1 antibody reduced transmission compared to that which occurred in transwells with an irrelevant control antibody.

These studies demonstrate a role for the β-integrin CD18 in transmission of HIV-1 infected cells across the cervical epithelium in vitro. The levels of blocking observed in vitro for anti-ICAM-1 have previously correlated to a highly significant reduction in cell-associated HIV-1 transmission using an in vivo HuPBL-SCID mouse model. Therefore, the high degree of blocking observed in the experiments of these Examples 1-2 demonstrate utility of antibody to CD18 as an anti-HIV-1 microbicide.

CD18 is the common β2-subunit of the Leu-cam family of cell adhesion receptors expressed on leukocytes, which includes LFA-1 (CD11a/CD18, α₁β₂), Mac-1 (CD11b/CD18, α_{M}β₂), p150,95 (CD11c/CD18, α_{X}β₂), and CD11d/CD18 (α_{D}β₂) (Sanchez-Madrid, F., J. A. Nagy, E. Robbins, P. Simon, and T. A. Springer, 1983, A human leukocyte differentiation antigen family with distinct alpha-subunits and a common beta-subunit: the lymphocyte function-associated antigen (LFA-1), the C3bi complement receptor (OKM1/Mac-1), and the p150,95 molecule, J Exp Med 158:1785-1803). The observation that antibody to CD18 blocks transmission of cell-associated HIV-1 (e.g. FIGS. 1 and 2) is consistent with the well-established role of CD18 in monocyte and lymphocyte adhesion and transendothelial migration (Greenwood, J., Y. Wang, and V. L. Calder, 1995, Lymphocyte adhesion and transendothelial migration in the central nervous system: the role of LFA-1, ICAM-1, VLA-4 and VCAM-1. off, Immunology 86:408-15; Hakkert, B. C., T. W. Kuijpers, J. F. Leeuwenberg, J. A. van Mourik, and D. Roos, 1991, Neutrophil and monocyte adherence to and migration across monolayers of cytokine-activated endothelial cells: the contribution of CD18, ELAM-1, and VLA-4, Blood 78:2721-6; Meerschaert, J., and M. B. Furie, 1995, The adhesion molecules used by monocytes for migration across endothelium include CD11a/CD18, CD11b/CD18, and VLA-4 on monocytes and ICAM-1, VCAM-1, and other ligands on endothelium, J Immunol 154:4099-112; Meerschaert, J., and M. B. Furie, 1994, Monocytes use either CD11/CD18 or VLA-4 to migrate across human endothelium in vitro, J Immunol 152:1915-26; Muller, W. A., and S. A. Weigl, 1992, Monocyte-selective transendothelial migration: dissection of the binding and transmigration phases by an in vitro assay, J Exp Med 176:819-28; Parkos, C. A., C. Delp, M. A. Arnaout, and J. L. Madara, 1991, Neutrophil migration across a cultured intestinal epithelium, Dependence on a CD11b/CD18-mediated event and enhanced efficiency in physiological direction, J Clin Invest 88:1605-12; Schenkel, A. R., Z. Mamdouh, and W. A. Muller, 2004, Locomotion of monocytes on endothelium is a critical step during extravasation, Nat Immunol 5:393-400; te Velde, A. A., G. D. Keizer, and C. G. Figdor, 1987, Differential function of LFA-1 family molecules (CD11 and CD18) in adhesion of human monocytes to melanoma and endothelial cells, Immunology 61:261-7.)

Elsewhere, it has been observed that different anti-ICAM-1 antibodies that were shown to block adhesion of ICAM-1 and its ligands were able to reduce transmission of HIV-1, as indicated by p24 ELISA, to widely varying degrees. In these Examples, it has been demonstrated that both adhesion-blocking anti-CD18 clones tested, H52 (Hildreth, J. E., V. Holt, J. T. August, and M. D. Pescovitz, 1989, Monoclonal antibodies against porcine LFA-1: species cross-reactivity and functional effects of beta-subunit-specific antibodies, Mol Immunol 26:883-95) and 7E4 (Nortamo, P., M. Patarroyo, C. Kantor, J. Suopanki, and C. G. Gahmberg, 1988, Immunological mapping of the human leucocyte adhesion glycoprotein gp90 (CD18) by monoclonal antibodies, Scand J Immunol 28:537-46), were able to significantly block transmission of both HIV-1 and migration of PBMC from HIV-1 infected cultures (FIG. 2). At all concentrations tested, antibody from clone H52 blocked more efficiently than antibody from clone 7E4.

As observed, Fab of anti-CD18 clone 7E4 significantly reduced both transmission of HIV-1 and reduced the number of PBMC from HIV-1 infected cultures crossing the cervical epithelial monolayers, though at a level slightly less than that of the intact 7E4 antibody (FIG. 10). In contrast to the intact antibody used previously, the Fab are monovalent, and more similar in size to the scFv that would be produced by engineered lactobacilli. The small reduction in the level of blocking observed may be due to a reduction in binding ability caused by the enzymatic digestion used to produce the Fab. These results suggest that a monovalent, secreted anti-CD18 scFv may be used in reducing transmission of cell-associated HIV-1.

In order to determine whether the level of blocking by antibody could be improved by combining two different antibodies, the level of blocking achievable by mixing anti-ICAM-1 and anti-CD18 was compared to that observed for the same concentration of each antibody alone. Mixtures of anti-ICAM-1 and either anti-CD18 clone H52 or 7E4 significantly reduced migration of HIV-1 infected cells (FIGS. 11A-B, 5, and 12A-B) and transmission of HIV-1 (FIG. 12C-D), compared to corresponding concentrations of either antibody alone. This could indicate that both ICAM-1 and CD18 may be also be engaging other binding partners that are minor contributors to the migration of HIV-1 infected cells across the cervical epithelium, but because the greatest benefit was observed at antibody concentrations that are suboptimal for blocking with single antibodies, it is more likely that the antibody combination blocks the ICAM-1/CD18 interaction more efficiently. This enhancement of blocking of cell-associated HIV-1 transmission by a 50:50 mixture of antibodies to the adhesion receptor pair anti-ICAM-1 and anti-CD18 is notable because it suggests that a more pronounced effect may be achieved with a lower total amount of antibody. Increased efficacy at lower concentrations (e.g., a concentration in a range of about 1 to 5 micrograms) would be desirable in an antibody-based microbicide regardless of delivery system. In other work, our laboratory demonstrated that anti-ICAM-1 scFv secreted by lactobacilli and purified prior to use in in vitro assays can block both transepithelial migration of cells from HIV-1 infected cultures and transmission of HIV-1 when used at a concentration of 67 μg/ml. Concentrations of up to 5 μg/ml have been achieved (data not shown) in broth culture.

Notably, antibody to CD18 at 40 μg/ml completely blocked transmission of cell-associated HIV-1 in the Hu-PBL SCID mouse model (Table 1).

In a two-chamber system, both antibodies, and particularly anti-CD18, clearly reduced infection of the subepithelial PBMC placed in the lower chamber. Concentrations of antibody could be detected in the lower chamber of the transwell at approximately 10% of the concentration observed in the upper chamber (data not shown). Although the interruption of infection was not complete in the transwell assay, such levels of reduction in cell-associated transmission correlated with 100% protection in the Hu-PBL-SCID mouse model. Thus anti-CD18 would appear to be effective in blocking both cell-associated and cell-free virus transmission.

These results of the experimental Examples demonstrate the importance of CD18 in sexual-transmission of HIV-1. Antibody to CD18 shows utility as an anti-HIV-1 microbicide using a lactobacillus-based delivery system. In addition, antibody to CD18 used in combination with antibody to ICAM-1 has been shown to block transepithelial HIV transmission at an antibody concentration which is feasibly provided using lactobacillus-produced scFv in situ in the female genitourinary tract.

Microbicides to prevent HIV-1 transmission to women may play a valuable role in stemming the worldwide HIV epidemic. Based on the experimentation herein, the inventors consider antibodies herein to host cell adhesion molecules useable as an anti-HIV-1 microbicide. These experimental examples demonstrate that two different clones of antibody to the P integrin CD18 reduce both transmission of HIV-1 and migration of cells from infected cultures (p<0.01). In addition, a 50:50 mixture of anti-ICAM-1 and either clone of anti-CD18 reduced transmission of PBMC from HIV-1 infected cultures significantly (p<0.05) more than either antibody used alone at the same total antibody concentration. In vivo, both anti-CD18 and a 50:50 mixture of anti-CD18 and anti-ICAM-1 significantly reduced vaginal transmission of cell-associated HIV-1 in HuPBL-SCID mice. These results demonstrate the importance of CD18 in the transmission of cell-associated HIV-1. In addition, antibody to CD18 used in combination with antibody to ICAM-1 has been shown to block at a concentration which can be achieved using lactobacillus-produced scFv in vivo.

### Example 9

### Recombinant Bacteria

Additionally, as an alternative embodiment to protective antibodies, recombinant bacteria (such as, e.g., rCD54) can be used to express functional (agonist) or defective (antagonist) ligands. For example, the engagement of ICAM-1 on the surface of epithelial cells by LFA-1 or Mac-1 on the surface of infected lymphocytes or macrophages could be blocked by occupation of the ICAM-1 receptor by a soluble form of the LFA-1 or Mac-1 ligands, as long as the soluble ligands are in a monovalent form that would not be capable of cross-linking the ICAM-1 receptor. Such cross-linking disrupts the epithelium. Similarly, soluble ICAM-1 could block binding to LFA-1 or Mac-1 by the ICAM-1 receptor on the surface of epithelial cells.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of preventing an initial HIV infection in a human or animal, comprising: exposing an epithelium which may receive HIV exposure to one or more anti-CD18 or anti-CD11 antibodies, or both, wherein the step of exposing the epithelium to the antibodies precedes any establishment of an HIV infection, and establishment of an HIV infection is prevented.

2. The method of claim 1, wherein said HIV is cell-free HIV.

3. The method of claim 1, wherein said HIV is cell-associated HIV.

4. The method of claim 1, including exposing the epithelium to anti-ICAM antibodies in combination with the CD18 and/or CD11 antibodies.

5. The method of claim 1, including delivering the antibodies to the epithelium via at least one bacterial delivery system.

6. The method of claim 1, including delivering the antibodies to the epithelium via at least one lactobacillus delivery system.

7. The method of claim 6, wherein an amount of lactobacillus delivery system used is sufficient to express scFv, diabodies of scFv, triabodies of scFv, and/or tetramers of scFv-like molecules producible by bacteria in a concentration in a range of from 0.5 to 100 micrograms/ml.

8. The method of claim 7, wherein a concentration of scFv, diabodies of scFv, triabodies of scFv, and/or tetramers of scFv-like molecules ranges from about 0.5 to 100 micrograms/ml.

9. The method of claim 1, wherein a bacterial delivery system expresses scFv.

10-16. (canceled)

17. The method of claim 13, wherein transmission of HIV across the epithelium is reduced or prevented.

18. The method of claim 13, wherein sexual transmission of HIV is reduced or prevented.

19. The method of claim 13, including exposing the epithelium to anti-ICAM antibodies in combination with the CD18 and/or CDII antibodies.

20. The method of claim 13, including delivering the antibodies to the epithelium via at least one bacterial delivery system.

21-29. (canceled)

30. A microbicide comprising: one or more anti-CD18 or anti-CDI 1 antibodies, or both.

31. The microbicide of claim 30, wherein the antibodies are expressed by a bacterial delivery system as scFv.

32. The microbicide of claim 30, wherein the antibodies are deliverable to a to-be-protected epithelium via at least one lactobacillus bacterial vehicle.

33-35. (canceled)

36. A method of constructing a microbicide, comprising:
(A) fusing an antigen-binding variable region of a light chain and an antigen-binding variable region of a heavy chain to a linker protein to form a single chain antibody (scFv), wherein the antibody molecule may be the same or different and is selected from anti-CD11 and anti-CD18;
(B) establishing conditions in which a bacterial delivery system expresses the single chain antibody of step (A) in an environment including an epithelium to be protected from HIV transmission.

37-38. (canceled)

39. An expression product that blocks transepithelial transmission of HIV-I, the expression product selected from the group consisting of: anti-CD18; anti-CDII; and a ligand expressed by a recombinant bacteria.
